(19) United States
(12) Patent Application Publication
(10) Pub. No.: US 2011/0265798 A1
(43) Pub. Date: Nov. 3, 2011

(54) MEDICAL DEVICE TUBE HAVING SPACED LUMENS AND AN ASSOCIATED PORTED ADAPTER

(75) Inventors: Seamus Maguire, Athlone (IE); Roger Harrington, Athlone (IE); Brian Ledwith, Co. Longford (IE)

(73) Assignee: Nellcor Puritan Bennett LLC, Boulder, CO (US)

(21) Appl. No.: 12/771,516

(22) Filed: Apr. 30, 2010

(51) Int. Cl. A61M 16/04

(52) U.S. Cl. 128/207.14

(57) ABSTRACT

Various embodiments of a tracheal tube having spaced lumens and an associated ported adapter are provided. The lumens of the tracheal tube are spaced around the circumference of the tracheal tube to facilitate evacuation (e.g., suctioning and blowing) and other applications at various locations around the circumference of the tracheal tube. The ported adapter includes lumen extensions that may be inserted into the lumens of the tracheal tube, thereby forming a connection between the ported adapter and the tracheal tube. More specifically, hollow ports extending through the lumen extensions and the body of the ported adapter facilitate connection of the lumens of the tracheal tube with external equipment such as, for example, evacuation equipment for suctioning and blowing into and out of the lumens of the tracheal tube.

### BACKGROUND

The present disclosure relates to tracheal tubes used in medical applications, and more particularly, to tracheal tubes having spaced lumens and associated ported adapters for connecting the tracheal tubes to cooperative devices, such as evacuation systems, ventilators, and so forth.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air, food, fluids, or other substances into and out of the patient. For example, medical devices, such as tracheal tubes, may be used to control the flow of air or other gases through a trachea of a patient. Such tracheal tubes may include endotracheal tubes (ETTs), tracheostomy tubes, or transtracheal tubes. In many instances, it is desirable to provide a seal between the outside of the tube or device and the interior of the passage in which the tube or device is inserted, such as the trachea. In this way, substances can only flow through the passage via the tube or other medical device inserted in the tube, allowing a medical practitioner to maintain control over the type and amount of substances flowing into and out of the patient. In addition, a high-quality seal against the tracheal passageway allows a ventilator to perform efficiently.

In many instances, it is also desirable to manage the accumulation of subglottic secretions (e.g., mucus) around the seal (e.g., a cuff) or elsewhere via removal through external suctioning. It may also be desirable to provide for administration of antibiotics and other medicaments in the same region. These subglottic secretions are undesirable as they may contain bacteria that may cause infection if left to grow. In some cases, these subglottic secretions may cause ventilator-associated pneumonia (VAP) due to bacterial colonization of the lower respiratory airways.

As such, the tracheal tubes may include one or more lumens extending axially through walls of the tracheal tubes. These lumens enable various auxiliary applications (e.g., tubes for enabling suctioning and blowing, cameras, devices for monitoring pressure, temperature, and other parameters, and so forth) to be introduced at various locations along the tracheal tubes. However, conventional tracheal tubes may not be configured to adapt to changes in patient orientation. More specifically, for example, the lumens of the tracheal tubes may only be aligned with locations requiring suctioning or blowing when the patient is oriented in a specific manner. As such, these tracheal tubes may only work efficiently if the patient is immobile and inclined at a certain attitude. Moreover, conventional evacuation and other lines may require piercing the tube wall to link a small external tube to the lumen. This can be time consuming, and where two or more lumens are present, can lead to a relatively confusing set of tubes and fittings.

### BRIEF DESCRIPTION OF THE DRAWINGS

Advantages of the disclosed techniques may become apparent upon reading the following detailed description and upon reference to the drawings in which:

FIG. 1 is a perspective view of an exemplary tracheal ventilation system equipped with a ported adapter;

FIG. 2 is a cross-sectional view of an exemplary endotracheal tube of the tracheal ventilation system of FIG. 1;

FIG. 3 is a perspective view of an exemplary ported adapter, ventilator connector, and endotracheal tube;

FIG. 4 is a side view of an exemplary ported adapter;

FIG. 5 is a partial side view of an exemplary ported adapter;

FIG. 6 is a partial cross-sectional side view of an exemplary ported adapter having lumen extensions with tapered walls; and

FIG. 7 is a side view of an exemplary ported adapter and ventilator connector.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As discussed in detail below, various embodiments are provided of a tracheal tube having equally spaced lumens and an associated ported adapter. The lumens of the tracheal tube may be spaced around the circumference of the tracheal tube to facilitate evacuation (e.g., suctioning and blowing) and other applications at various locations around the circumference of the tracheal tube. The ported adapter includes lumen extensions that may be inserted into the lumens of the tracheal tube, thereby forming a connection between the ported adapter and the tracheal tube lumens. More specifically, hollow ports extending through the lumen extensions and the body of the ported adapter facilitate connection of the lumens of the tracheal tube with external equipment such as, for example, evacuation equipment for suctioning and blowing into and out of the lumens of the tracheal tube.

The devices and techniques provided herein enable evacuation (e.g., suctioning and blowing) and other applications to be performed at various locations around the circumference of the tracheal tube. As such, problems associated with patient orientation and blockage of certain lumens will be reduced. In addition, using a removable ported adapter to connect the spaced lumens of the tracheal tube with external equipment enables different ported adapters to be used with the tracheal tube. For example, certain ported adapters may include ports for each of the lumens of the tracheal tube, whereas other ported adapters may only include ports for a few (or even one) of the lumens of the tracheal tube.

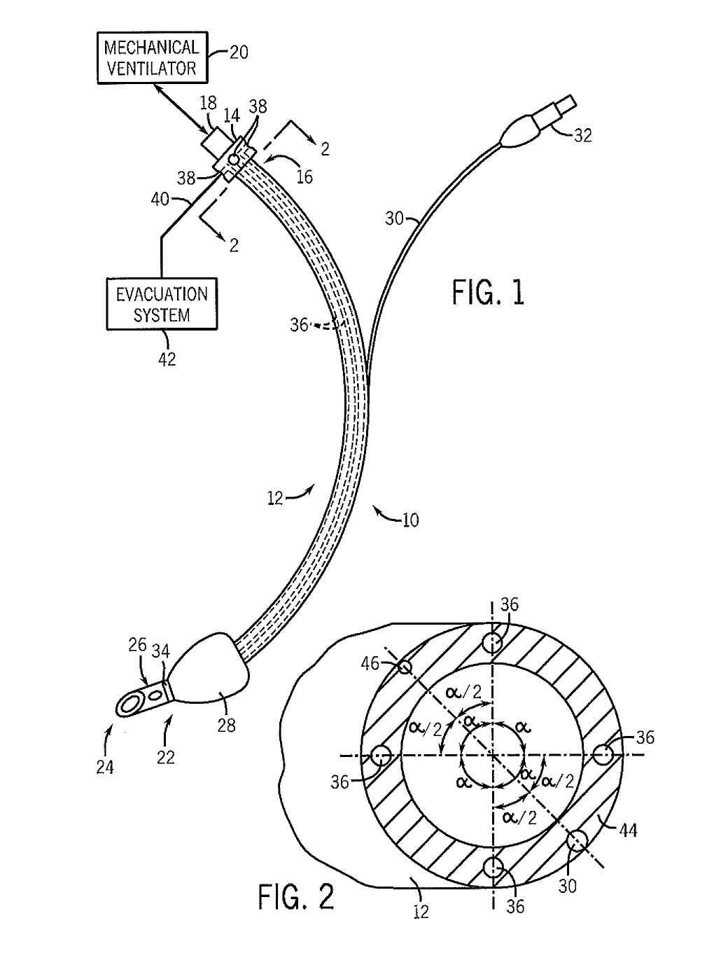

Turning now to the drawings, FIG. 1 is a perspective view of an exemplary tracheal ventilation system 10 equipped with a ported adapter. More specifically, the tracheal ventilation system of FIG. 1 illustrates an endotracheal tube 12 attached to a ported adapter 14. Although illustrated as an endotracheal tube 12, the ported adapter 14 may be used with tracheostomy tubes, transtracheal tubes, or any other suitable tracheal tubes. In the illustrated embodiment, the ported adapter 14 is attached to a proximal end 16 of the endotracheal tube 12, and a ventilator connector 18 is attached to (or integrally formed with) the ported adapter 14. The ventilator connector 18 may be attached to a mechanical ventilator 20 during operation. A distal end 22 of the endotracheal tube 12 terminates in an opening 24 and may be placed in a patient's trachea during operation to maintain airflow to and from the patient's lungs. A Murphy's eye 26 may be located on the endotracheal tube 12 near the opening 24 to prevent airway occlusion when the endotracheal tube 12 is improperly placed within the patient's trachea.

As illustrated, an inflation cuff 28 that may be inflated to seal against the walls of a body cavity (e.g., a trachea) may be attached along and above the distal end 22 of the endotracheal tube 12. The inflation cuff 28 may be inflated via an inflation lumen 30 connected to a fixture 32. A shoulder 34 of the inflation cuff 28 may secure the inflation cuff 28 to the endotracheal tube 12. In certain embodiments, the shoulder 34 may be folded up inside a lower end of the inflation cuff 28, although various alternative structures are common in the art. As illustrated, the endotracheal tube 12 also includes a plurality of lumens 36 that extend from locations on the endotracheal tube 12 above the inflation cuff 28. For example, each of the lumens 36 may be associated with notches on a proximal side of the inflation cuff 28. The lumens 36 illustrated in this embodiment are equally spaced around the circumference of the endotracheal tube 12. As described in greater detail below, the lumens 36 may be connected with corresponding ports 38 of the ported adapter 14. It should be noted, however, that the lumens 36 need not be equally spaced in all applications, and indeed may be provided for different purposes than evacuation.

One or more of the ports 38 may be connected to an external evacuation tube 40 and an evacuation system 42 used for the removal and introduction of suctioned and blown fluids. Where the lumens 36 are at spaced locations around the circumference of the endotracheal tube 12, the lumens 36 facilitate evacuation at various possible orientations of the patient (e.g., sitting, reclined, and lying on the back, chest, or side). In certain embodiments, to prevent suctioning or blowing from lumens 36 not requiring suctioning or blowing, a method of sequential bi-directional flow through the lumens 36 may be employed. The bi-directional flow may allow any blockage to be expelled from the affected lumen 36. This method of sequential bi-directional flow may employ a powered valve (or manual valve) allowing alternating bi-directional flow to each lumen 36 in turn. Moreover, in certain embodiments, a more complex method may use feedback sensors to, for example, identify when a specific lumen 36 is producing suctionable secretions and, when appropriate, commence suctioning of the corresponding port 38 of the ported adapter 14. As such, only lumens 36 that are ideally placed for suctioning or blowing will be subject to evacuation. Similarly, pressure sensors may be used to detect pressure changes due to blockages, and to instigate a suction/blow cycle until normal pressures resume.

Furthermore, the plurality of lumens 36 and ports 38 may be used for various other applications of the endotracheal tube 12. For example, in addition to being used for evacuation purposes, the lumens 36 and ports 38 may be used to introduce other devices and/or fluids (e.g., cameras, sensors, medicaments, and so forth) into the patient's trachea.

FIG. 2 is a cross-sectional view of an exemplary endotracheal tube 12 of the tracheal ventilation system 10 of FIG. 1. As illustrated, the endotracheal tube 12 may include one or more lumens 36 spaced around the circumference of the annular wall 44 of the endotracheal tube 12. Although illustrated as having four equally spaced lumens 36, the endotracheal tube 12 may include any number of spaced lumens 36, the spacing of which may be adapted to suit a particular application. For example, the endotracheal tube 12 may include 1, 2, 3, 4, 5, 6, 7, 8, 9, 10, 11, 12, or even more lumens 36. In general, the spaced lumens 36 may be separated from one another around the circumference of the wall 44 of the endotracheal tube 12 by an angle α that is approximately equal to 360 degrees divided by the number of lumens 36. For example, in the illustrated embodiment, the four equally spaced lumens 36 are separated from one another around the circumference of the wall 44 of the endotracheal tube 12 by 90 degrees (i.e., 360 degrees divided by 4).

As noted above, the spaced lumens 36 may be used for various applications of the endotracheal tube 12. For example, the lumens 36 may be used for suctioning, blowing, introducing cameras, introducing sensors, introducing medicaments, and so forth. Indeed, the lumens 36 are multifunctional, facilitating any number of suitable functions of the endotracheal tube 12. As described above, in the illustrated embodiment, because the lumens 36 are spaced around the circumference of the endotracheal tube 12, the lumens 36 facilitate better suctioning and blowing in all patient orientations, while also facilitating removal of blockages.

In addition to the spaced, multifunctional lumens 36, in certain embodiments, the endotracheal tube 12 may include the inflation lumen 30 for inflating the inflation cuff 28, and an X-ray lumen 46, which may be filled with a fluid such as barium to render the structure visible in X-ray images. In general, the inflation lumen 30 and the X-ray lumen 46 may be smaller than the lumens 36. For example, the lumens 36 may be approximately 1.5-3.0 mm in diameter, whereas the inflation lumen 30 and the X-ray lumen 46 may be approximately 0.8-1.2 mm and 0.3-0.8 millimeter in diameter, respectively. By way of comparison, in certain embodiments, the wall 44 of the endotracheal tube 12 may have a width of approximately 2.0-4.0 mm, depending upon the particular application. It should be noted that certain of the conventional lumens, such as the inflation lumen 30, may also be ported though the ported adapter 14.

In certain embodiments, both the inflation lumen 30 and the X-ray lumen 46 may be offset from the equally spaced, multifunctional lumens 36 around the circumference of the wall 44 of the endotracheal tube 12. For example, as illustrated in FIG. 2, the inflation lumen 30 and the X-ray lumen 46 may be located exactly halfway between adjacent equally spaced, multifunctional lumens 36 around the circumference of the wall 44 of the endotracheal tube 12 (e.g., at an arc angle of α/2 from the equally spaced, multifunctional lumens 36). However, in other embodiments, the inflation lumen 30 and the X-ray lumen 46 may be located anywhere between adjacent equally spaced, multifunctional lumens 36 around the circumference of the wall 44 of the endotracheal tube 12.

The endotracheal tube 12 may be manufactured using extrusion techniques and may be made from any material suitable for use in tracheal tubes. For example, in certain embodiments, the endotracheal tube 12 may be made of polyurethane, polyvinyl chloride (PVC), polyethylene teraphthalate (PETP), low-density polyethylene (LDPE), polypropylene, silicone, neoprene, polytetrafluoroethylene (PTFE), or polyisoprene. Moreover, the lumens 36 may be specifically dedicated to certain types of applications (e.g., inflation, evacuation, etc.), or the tubes may be designed with a number of “multi-purpose” lumens, as set forth in U.S. patent application Ser. No. 12/750,789, filed on Mar. 31, 2010, and entitled “Tracheal Tube with Scaffolding-Supported Wall,” which is hereby incorporated by reference.

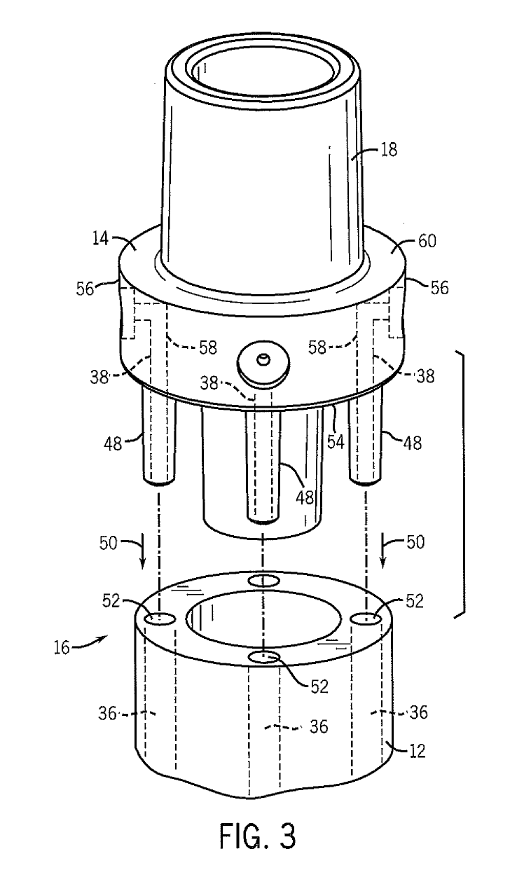

As described above, the ported adapter 14 of FIG. 1 is configured to connect with the endotracheal tube 12 such that the lumens 36 of the endotracheal tube 12 are connected with the ports 38 of the ported adapter 14. In turn, the ports 38 of the ported adapter 14 may be used to connect with external equipment for introducing various applications (e.g., evacuation, cameras, sensors, medicaments, and so forth). FIG. 3 is a perspective view of an exemplary ported adapter 14, ventilator connector 18, and endotracheal tube 12. In particular, FIG. 3 illustrates how the ported adapter 14, ventilator connector 18, and the endotracheal tube 12 connect with each other. As illustrated, the ported adapter 14 includes one or more lumen extensions 48 (e.g., prongs) that correspond to the lumens 36 of the endotracheal tube 12. For example, in certain embodiments, the lumen extensions 48 of the ported adapter 14 may have generally circular outer diameters that correspond to generally circular inner diameters of the lumens 36 of the endotracheal tube 12. However, in other embodiments, the particular geometries of the lumens extensions 48 and the lumens 36 may vary between applications. Regardless, the outer cross-sectional geometries of the lumen extensions 48 will generally correspond to the inner cross-sectional geometries of the lumens 36.

As illustrated by the arrows 50 in FIG. 3, the ported adapter 14 may be slid into place with respect to the endotracheal tube 12 such that the lumen extensions 48 slide or are pressed into openings 52 of the lumens 36 at the proximal end 16 of the endotracheal tube 12. Once the lumen extensions 48 of the ported adapter 14 are in place within the lumens 36 of the endotracheal tube 12, the hollow ports 38 within the lumen extensions 48 will generally act as an extension of the lumens 36 through the ported adapter 14.

In certain embodiments, the hollow ports 38 may extend from a distal surface 54 of the ported adapter 14 to an outer circumferential wall 56 of the ported adapter 14. As illustrated, the distal surface 54 of the ported adapter 14 is adjacent to and orthogonal with the outer circumferential wall 56 of the ported adapter 14. A 90-degree bend 58 in each of the hollow ports 38 enable the hollow ports 38 to bend from the distal surface 54 of the ported adapter 14 to the outer circumferential wall 56 of the ported adapter 14. Although illustrated as extending from the distal surface 54 of the ported adapter 14 to the adjacent outer circumferential wall 56 of the ported adapter 14, in other embodiments, the hollow ports 38 may extend from the distal surface 54 of the ported adapter 14 to a proximal surface 60 of the ported adapter 14, as described in greater detail below. As illustrated, the proximal surface 60 of the ported adapter 14 is opposite to the distal surface 54 of the ported adapter 14. It may also be desirable to provide angled or inclined walls on the ported adapter 14, with the polls 38 terminating in that wall, to allow for easy access to the ports 38, while avoiding any sharp angles within the ported adapter 14.

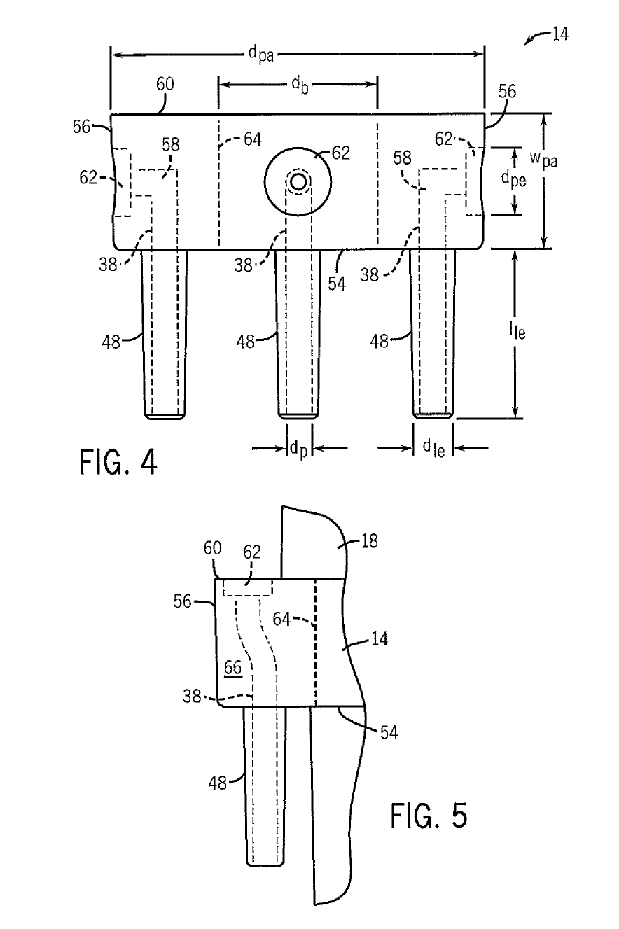

FIG. 4 is a side view of an exemplary ported adapter 14. As described above, the lumen extensions 48 extend from the distal surface 54 of the ported adapter 14 such that the lumen extensions 48 may be inserted into the lumens 36 of the endotracheal tube 12. In certain embodiments, the lumen extensions 48 may extend by a substantial distance from the distal surface 54 of the ported adapter 14. For example, as illustrated, the lumen extensions 48 may have a length lₗₑ, of approximately 5-10 mm. By way of comparison, in certain embodiments, the width wₚₐ of the ported adapter 14 may also be approximately 5-10 mm. However, in other embodiments, the lumen extensions 48 may have a longer length lₗₑ (e.g., approximately 10-15 mm or greater) or a shorter length lₗₑ (e.g., approximately 1-5 mm). In general, however, the lumen extensions 48 may be long enough to ensure that the lumen extensions 48 remain in place within their respective lumens 36 of the endotracheal tube 12.

As described above, each lumen 36 of the endotracheal tube 12 may have an inner diameter of known dimensions, such as approximately 1.5-3.0 mm. As such, each lumen extension 48 of the ported adapter 14 has a corresponding outer diameter dₗₑ of the same or slightly larger dimension, such that the lumen extensions 48 may be interference fit within the lumens 36, ensuring that the lumen extensions 48 remain in place when attached to the lumens 36. As illustrated, the inner diameter dₚ of the ports 38 have a slightly smaller diameter than the outer diameter dₗₑ of lumen extensions 48. However, as described below, in certain embodiments, the lumen extensions 48 may include a tapered wall that enables the inner diameter dₚ of the ports 38 to be substantially similar to the inner diameter of the lumens 36 of the endotracheal tube 12. It is presently contemplated that the ported adapter 14 may simply be fitted to the endotracheal tube 12 and may remain in place by the interference fit of the lumen extensions 48 within the lumens 36. However, where desired, adhesive or other bonding techniques may be used, particularly where differently configured lumen extensions 48 are employed that may not provide the pull-out resistance desired.

As illustrated in FIG. 4, each hollow port 38 has a substantially constant inner diameter dₚ from the distal surface 54 of the ported adapter 14 to the outer circumferential wall 56 of the ported adapter 14. However, in certain embodiments, each hollow port 38 may terminate in a port exit 62, which consists of a substantially larger cross-sectional area than its respective hollow port 38. For example, the port exits 62 may have an inner diameter of approximately 3-8 mm. The substantially larger port exits 62 facilitate connection of the ports 38 (and associated lumens 36) with external equipment, such as evacuation systems, cameras, sensors, and so forth.

As illustrated in FIG. 4, the ported adapter 14 may also have an interior bore 64, within which the ventilator connector 18 may fit, as described in greater detail below. In certain embodiments, the interior bore 64 may have an inner diameter d_{b} of approximately 5-10 mm, as compared to the outer dₚₐ of the ported adapter 14, which may be approximately 15-25 mm. It should be noted that the dimensions of the ported adapter 14 described above with respect to FIG. 4 are merely exemplary and are not intended to be limiting.

Although illustrated in FIGS. 1, 3, and 4 as extending from the distal surface 54 of the ported adapter 14 to the adjacent outer circumferential wall 56 of the ported adapter 14, in certain embodiments, the hollow ports 38 may instead extend from the distal surface 54 of the ported adapter 14 to the opposite proximal surface 60 of the ported adapter 14. For example, FIG. 5 is a partial side view of an exemplary ported adapter 14. As illustrated, the hollow ports 38 extend from the distal surface 54 of the ported adapter 14 to the opposite proximal surface 60 of the ported adapter 14. However, in certain embodiments, the hollow ports 38 may not extend orthogonally through a main body 66 of the ported adapter 14 from the distal surface 54 to the proximal surface 60. Rather, the hollow ports 38 may follow a slightly curved or straight angular path such that the hollow ports 38 extend from the distal surface 54 of the ported adapter 14 to the proximal surface 60 of the ported adapter 14 at a slight angle. The slight curvature or angle of the hollow ports 38 allows the port exits 62 in the proximal surface 60 to be located closer to the outer circumferential wall 56 of the ported adapter 14. Having the port exits 62 closer to the outer circumferential wall 56 of the ported adapter 14 allows more surface area on the proximal surface 60 of the ported adapter 14 for the ventilator connector 18 to abut the ported adapter 14 while still enabling the port exits 62 to connect to external equipment.

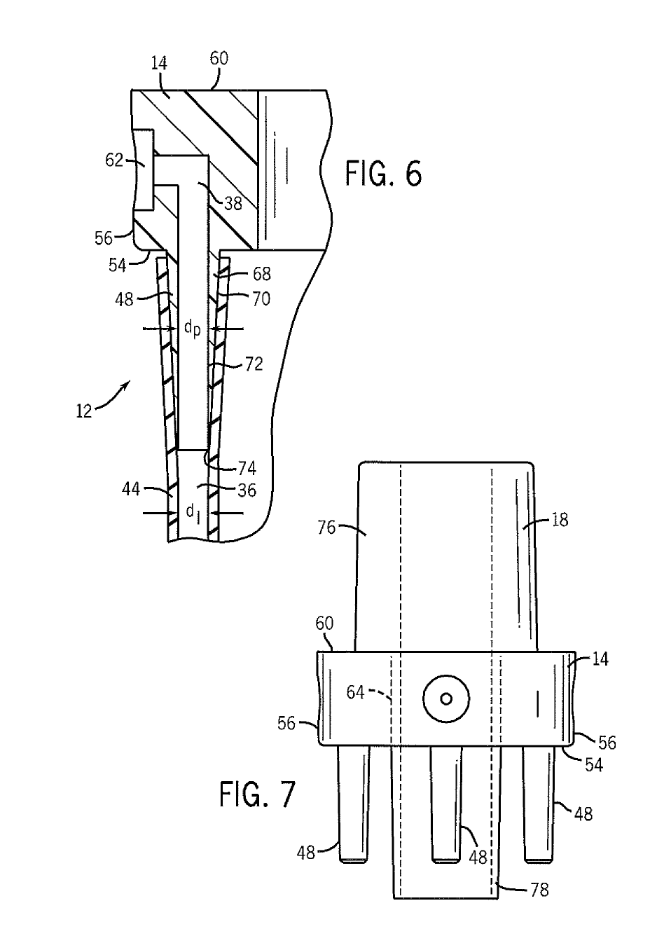

As described above, in certain embodiments, the lumen extensions 48 of the ported adapter 14 may include tapered walls. For example, FIG. 6 is a partial cross-sectional side view of an exemplary ported adapter 14 having lumen extensions 48 with tapered walls 68. The embodiment of the ported adapter 14 illustrated in FIG. 6 is similar to the embodiments illustrated in FIGS. 1, 3, and 4. However, the lumen extension 48 of the ported adapter 14 illustrated in FIG. 6 includes a tapered exterior surface 70, which meets with an interior surface 72 at a distal point 74 of the lumen extension 48. The interior surface 72 of the lumen extension 48 is generally orthogonal to the distal surface 54 of the ported adapter 14, whereas the tapered exterior surface 70 extends from the distal surface 54 of the ported adapter 14 at an angle toward the distal point 74. As such, the tapered wall 68 of the lumen extension 48 enables the lumen extension 48 to remain connected to the lumen 36 of the endotracheal tube 12, but also to have the inner diameter dₚ of the hollow port 38 within the lumen extension 48 approximately equal to the inner diameter dₗ of the lumen 36. More specifically, when the lumen extension 48 of the ported adapter 14 is inserted into the lumen 36 of the endotracheal tube 12, the tapered wall 68 causes the wall 44 of the endotracheal tube 12 to expand slightly in a radial direction, thereby enhancing the interference fit of the lumen extension 48 within the lumen 36.

As described above, the ported adapter 14 may be configured to connect with a ventilator connector 18, which in turn may be used to connect to a ventilation system. For example, FIG. 7 is a side view of an exemplary ported adapter 14 and ventilator connector 18. As illustrated, the ventilator connector 18 may fit within the interior bore 64 of the ported adapter 14. The ventilator connector 18 may include a proximal section 76 and a distal section 78, both having an annular cross-section.

In certain embodiments, the distal section 78 of the ventilator connector 18 may have a generally circular cross-section such that the circular distal section 78 of the ventilator connector 18 may fit within the circular interior bore 64 of the ported adapter 14. As illustrated, when inserted into the interior bore 64 of the ported adapter 14, the distal section 78 may extend beyond the lumen extensions 48 of the ported adapter 14. As such, the distal section 78 of the ventilator connector 18 may be inserted into the cannula of the endotracheal tube 12, thereby connecting the ventilator connector 18 with the cannula of the endotracheal tube 12.

Conversely, in certain embodiments, the proximal end 76 of the ventilator connector 18 may have a cross-sectional area that is larger than that of the interior bore 64 of the ported adapter 14. As illustrated, when the distal end 78 of the ventilator connector 18 is inserted into the interior bore 64 of the ported adapter 14, the proximal end 76 may abut the proximal surface 60 of the ported adapter 14. As such, the ventilator connector 18 may be prevented from sliding further into the ported adapter 14 or the endotracheal tube 12. Once the ventilator connector 18 is inserted into the ported adapter 14 and the endotracheal tube 12, the proximal end 76 of the ventilator connector 18 may be connected to an external ventilation system, enabling ventilation of the patient's trachea.

It should be noted that, in certain embodiments, the ported adapter 14 and the ventilator connector 18 may not be separate components. Rather, the ported adapter 14 and the ventilator connector 18 may be integrated into a single adapter/connector piece. In other words, the ported adapter 14 may be designed as a standard ventilator connector. In certain embodiments, the ported adapter 14 and the ventilator connector 18 (as well as the integrated adapter/connector piece) may be made using injection molding techniques, and may be made from polypropylene, polyvinyl chloride (PVC), polyethylene teraphthalate (PETP), acrylonitrile butadiene styrene (ABS), or any other suitable materials.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A tracheal ventilation system, comprising:
a ported adapter having a main body, a lumen extension protruding from a first external surface of the main body, and a hollow port extending through the lumen extension and the main body to a second external surface of the main body.

2. The tracheal ventilation system of claim 1, wherein the first external surface of the main body is adjacent to the second external surface of the main body.

3. The tracheal ventilation system of claim 1, wherein the first external surface of the main body is opposite to the second external surface of the main body.

4. The tracheal ventilation system of claim 3, wherein the hollow port extends from the first surface of the main body to the second surface of the main body at an angle with respect to both the first and second surfaces of the main body.

5. The tracheal ventilation system of claim 1, wherein the lumen extension comprises a tapered wall.

6. The tracheal ventilation system of claim 1, wherein the lumen extension is generally circular.

7. The tracheal ventilation system of claim 1, comprising a ventilation system connector having an annular cross-section, wherein the annular cross-section of the ventilation system connector fits securely within a bore of the ported adapter.

8. The tracheal ventilation system of claim 1, wherein the hollow port is substantially larger at the second external surface of the main body than at the first external surface of the main body.

9. A tracheal ventilation system, comprising:
a tracheal tube having an annular tubing wall and a plurality of lumens spaced around a circumference of the annular tubing wall; and
a ported adapter having a main body, a plurality of lumen extensions protruding from a first external surface of the main body, and a plurality of hollow ports extending through the lumen extensions and the main body to a second external surface of the main body, wherein the plurality of lumen extensions fit securely within the plurality of lumens.

10. The tracheal ventilation system of claim 9, wherein the first external surface of the main body is adjacent to the second external surface of the main body.

11. The tracheal ventilation system of claim 9, wherein the first external surface of the main body is opposite to the second external surface of the main body.

12. The tracheal ventilation system of claim 11, wherein the hollow port extends from the first surface of the main body to the second surface of the main body at an angle with respect to both the first and second surfaces of the main body.

13. The tracheal ventilation system of claim 9, wherein the lumen extensions comprise tapered walls.

14. The tracheal ventilation system of claim 13, wherein the lumen extensions comprise interior surfaces having a first substantially constant inner diameter that is approximately equal to a second substantially constant inner diameter of the lumens.

15. The tracheal ventilation system of claim 9, comprising a ventilation system connector having an annular cross-section, wherein the annular cross-section of the ventilation system connector fits securely within a bore of the ported adapter.

16. A tracheal ventilation system, comprising:
a tracheal tube having an annular tubing wall and a plurality of lumens spaced around a circumference of the annular tubing wall;
a ported adapter having a main body, a plurality of lumen extensions protruding from a first external surface of the main body, and a plurality of hollow ports extending through the lumen extensions and the main body to a second external surface of the main body, wherein the plurality of lumen extensions fit securely within the plurality of lumens; and
a ventilation system connector having an annular cross-section, wherein the annular cross-section of the ventilation system connector fits securely within a bore of the ported adapter.

17. The tracheal ventilation system of claim 16, wherein the first external surface of the main body is adjacent to the second external surface of the main body.

18. The tracheal ventilation system of claim 16, wherein the first external surface of the main body is opposite to the second external surface of the main body.

19. The tracheal ventilation system of claim 16, wherein the lumen extensions comprise tapered walls.

20. The tracheal ventilation system of claim 19, wherein the lumen extensions comprise interior surfaces having a first substantially constant inner diameter that is approximately equal to a second substantially constant inner diameter of the lumens.
